(19) United States
(12) Patent Application Publication
(10) Pub. No.: US 2020/0089733 A1
(43) Pub. Date: Mar. 19, 2020

(54) SENSOR PLACEMENT METHOD FOR REDUCING UNCERTAINTY OF STRUCTURAL MODAL IDENTIFICATION

(71) Applicant: Dalian University of Technology, Dalian, Liaoning (CN)

(72) Inventors: Tinghua YI, Dalian, Liaoning (CN); Xueyang PEI, Dalian, Liaoning (CN); Chunxu QU, Dalian, Liaoning (CN); Hongnan LI, Dalian, Liaoning (CN)

(21) Appl. No.: 16/342,951

(22) PCT Filed: Jun. 4, 2018
(86) PCT No.: PCT/CN2018/089749

§ 371 Date: Apr. 17, 2019

(51) Int. Cl. G06F 17/18; A61B 5/00

(52) U.S. Cl. CPC G06F 17/18; A61B 5/0024

(57) ABSTRACT

Sensor placement for structural health monitoring and sensor placement method for reducing uncertainty of structural modal identification. Influences of structural model error and measurement noise on measured responses are separated. Structural stiffness variation is used as model error, and Gaussian noise is used as measurement noise. Monte Carlo method simulates a large number of possible cases, and structural mode shape matrices under each model error condition are obtained. Conditional information entropy index quantifies and calculates uncertainty of identified modal parameter results. Conditional information entropy index solves the problem of uncertain Fisher information matrix, which cannot be solved by traditional information entropy method. Optimal sensor placement corresponds to maximum conditional information entropy index value. The sensor placement method considers influences of structural model error and measurement noise on structural modal identification, which is helpful for improving accuracy of structural modal parameter identification.

### TECHNICAL FIELD

The presented invention belongs to the technical field of sensor placement for structural health monitoring. Considering the influences of structural model error and measurement noise on the measured response data, a sensor placement method using conditional information entropy as the criterion index is proposed.

### BACKGROUND

Sensor placement plays an important role in structural health monitoring. The quantity and quality of measured data directly affect the operational performance of the structural health monitoring system. How to use a limited number of sensors to obtain as much useful information as possible is a problem to be considered in the optimal sensor placement. In the field of structural health monitoring, structural modal parameter identification has very important significance in structural state identification, finite element model updating and structural damage identification. The structural modal coordinate of the structure is linearly related to the response of the structure, so the modal coordinates is generally used as the modal parameters to be identified. Based on the method of structural modal coordinate recognition, there have been many researches: the effective independent method that makes the mode shape matrix independent and distinguishable; the modal kinetic energy method that comprehensively considers the mass matrix and the mode shape matrix; the time domain information entropy method that quantifies the uncertainty of the modal parameter identification results; the frequency domain information entropy method that focus on structural frequency domain parameter (i.e. frequency, damping ratio, mode shape) identification, and the like. For these methods, most assume that the prediction error between the measured and actual structural response is Gaussian noise.

At present, the sensor placement methods are more directed to the arrangement of the acceleration (displacement) sensors, and these methods can be well applied to the acquisition of structural modal parameter information. Existing sensor placement methods, considering the uncertainty of structural modal parameter identification, perform well in the accuracy of modal parameter identification. In engineering practice, acceleration (displacement) sensors are widely used, and modal parameters are critical to the state assessment of the structure. The effect of modal parameter identification is affected by the combination of structural model error and measurement noise. Existing sensor placement methods generally only consider measurement noise. The sensor placement method for modal parameter identification which comprehensively considers structural model error and measurement noise proposed by the invention has great research prospects in structural health monitoring.

### SUMMARY

In the present invention, the structural model error and the measurement noise are separately considered, and a new conditional information entropy criterion is proposed to quantify the uncertainty of the modal parameter identification. The random variation of the structural stiffness matrix is used to simulate the model error of the structure; the measurement noise is Gaussian noise. The conditional information entropy is used to quantify and calculate the uncertainty of the identified modal coordinate parameter results. When the value of the conditional information entropy is the smallest, the uncertainty of the identification result is the smallest, and the corresponding positions are the optimal sensor placement. The Monte Carlo method is used to calculate the conditional information entropy. The introduction of the concept of redundancy threshold can effectively avoid obtaining redundancy modal information caused by adjacent sensor positions. A sequential placement algorithm is proposed to guide the implementation of the sensor placement method.

A sensor placement method for reducing uncertainty of structural modal identification, the steps are as follows: establish the relationship between structural model error and measurement noise; conditional information entropy based sensor placement method.

1. Establish the Relationship Between Structural Model Error and Measurement Noise

Step 1.1: In structural health monitoring systems, the prediction error between the measured and actual structural response is due to two causes: model error and measurement noise, thereby establishing the following relationship:

y(t)=S(x(t,θ)+e(t,θ)) (1)

where: y(t)∈ [symbol] is a measured structural response of Ns degrees of freedom; N_{d} is a number of total degrees of freedom of the structure; S∈ [symbol] is a selection matrix for the sensor locations; θ∈ [symbol] is a modal parameter vector to be identified; e(t, θ)∈ [symbol] is a prediction error between the measured and actual structural response, which can be expressed as:

e(t,θ)=eₘₑₐ(t,θ)+e_{mod}(t,θ) (2)

where: eₘₑₐ(t,θ) is measurement noise; e_{mod} (t,θ) is the part of the prediction error caused by the structural model error.

Step 1.2: Define the form of the prediction error: the measurement noise is assumed to be a zero-mean Gaussian noise with a covariance matrix of Σₘₑₐ=diag(σ₁, . . . , σ_{N}_{_{d}}), σᵢ=σ₀; the structural model error is represented by the stiffness variation of the structure which is shown as:

$$Δ K = {\sum }_{j = 1}^{{N}_{e}} {β}_{j} {K}_{j} \qquad ( 3 )$$

where: Nₑ represents a number of structural element stiffness matrices; Kⱼ is the jth element stiffness matrix; βⱼ is a perturbation coefficient of the jth element stiffness matrix.

The change in the structural modal matrix is expressed as:

$$\begin{matrix}{ΔΦ}_{i} = [ {{\sum }_{r = 1}^{{N}_{d}}}_{r \neq i} \frac{- {Φ}_{r}^{T} {K}_{1} {Φ}_{i}}{{λ}_{r} - {λ}_{i}} {Φ}_{r} , … , {{\sum }_{r = 1}^{{N}_{d}}}_{r \neq i} \frac{- {Φ}_{r}^{T} {K}_{{N}_{e}} {Φ}_{i}}{{λ}_{r} - {λ}_{i}} {Φ}_{r} ] \\ {[ {β}_{1} , … , {β}_{{N}_{e}} ]}^{T} \\ = {E}_{i} β\end{matrix} \qquad ( 4 )$$

where: β is a perturbation coefficient vector of each element stiffness matrix, Eᵢ is a sensitivity coefficient matrix of the ith mode; ΔΦᵢ is a change of the ith mode shape vector; Φᵣ is a rth mode shape vector; λᵣ and λᵢ are the eigenvalues of the rth and ith mode respectively; the superscript T indicates transposition.

The mode shape changes of each order of the structure are expressed as:

ΔΦ=[E₁β,E₂β, . . . ,E_{N}_{ₘ}β] (5)

where: ΔΦ represents mode shape changes of each order mode of the structure; the subscript Nₘ indicates a corresponding modal order.

Step 1.3: Establish a measurement data expression that comprehensively considers the structural model error and measurement noise. Eq. (1) is rewritten as:

y(t)=S((Φ+[E₁β,E₂β, . . . ,E_{N}_{ₘ}β])θ+eₘₑₐ(t,θ)) (6)

where: Φ is a mode shape matrix calculated by the finite element model used in the structure. As seen from Eq. (6), the prediction error between the measured response and the actual response is represented by the two parts caused by the model error and the measurement noise.

2. Conditional Information Entropy Based Sensor Placement Method

Step 2.1: Use the probability density function to represent the uncertainty of the recognition result of modal coordinate parameters:

$$\begin{matrix}p ( θ \vert {Σ}_{mea} , D , β ) = c \frac{1}{{( \sqrt{2 π } {σ}_{0} )}^{{NN}_{s}}} exp [ - \frac{{NN}_{s}}{2 {σ}_{0}^{2}} J ( θ \vert D , β ) ] π ( θ \vert β ) & ( 7 ) \\ J ( θ \vert {Σ}_{mea} , D , β ) = \frac{1}{{NN}_{s}} {\sum }_{k = 1}^{N} {[ {y}_{k} - Sx ( θ , k \vert β ) ]}^{T} [ {y}_{k} - Sx ( θ , k \vert β ) ] & ( 8 )\end{matrix}$$

where: p(θ|Σₘₑₐ, D, β) indicates conditional probability density function; π(θ|β) is a prior distribution of modal coordinate parameters θ; c is a constant, ensuring that the integral summation value of Eq. (7) is 1; N is total number of samples; k represents sampling time.

Step 2.2: According to Eq. (8), the Fisher information matrix is obtained:

Q(S,θ₀|β)=(S(Φ±[E₁β,E₂β, . . . ,E_{N}_{ₘ}β]))^{T}(S(Φ±[E₁β,E₂β, . . . ,E_{N}_{ₘ}β])) (9)

where: Q(S, θ₀|β) is the Fisher information matrix.

Step 2.3: Derive the conditional information entropy for quantifying and calculating the uncertainty of the modal parameter identification.

h(S|ΣₘₑₐD,B)□∫_{β∈B}−ln[det(Q(S,θ₀|β))]π(β)dβ (10)

where: h(S|Σₘₑₐ, D, B) is conditional information entropy; B is a range of the perturbation coefficients.

Remove the negative sign to get the conditional information entropy index:

CIE(S)=∫_{β∈B} ln[det(Q(S,θ₀|β))]π(β)dβ (11)

Step 2.4: Establish the finite element model of structure, determine the candidate sensor placement positions; use Monte Carlo method to obtain the range of the perturbation coefficient B and the structural mode shape matrix in the corresponding situation; the initial number of selected sensors is 0.

Step 2.5: Whether to consider structural modal information redundancy: do not consider, continue to the next step; consider, jump to Step 2.9.

Step 2.6: Add one sensor location from the remaining candidate sensor positions to join the existing positions. Calculate the CIE(S) value, and the sensor location corresponding to the maximum value is selected.

Step 2.7: From the remaining candidate sensor positions, delete the selected sensor location. Check the remaining positions, if there is no remaining position, continue to the next step; if there are remaining positions, return to Step 2.6.

Step 2.8: Get the final sensor placement and jump out of the loop.

Step 2.9: If there are sensor locations too close, they contain similar structural modal information, resulting in redundancy of acquired structural modal information. Introduce the concept of structural modal information redundancy as:

$${γ}_{p , q} = 1 - \frac{{‖ {Φ}_{p} - {Φ}_{q} ‖}_{F}}{{‖ {Φ}_{p} ‖}_{F} + {‖ {Φ}_{q} ‖}_{F}} \qquad ( 12 )$$

where: γ_{p,q} represents a redundancy coefficient between the pth node position and the qth node position in the finite element structure; the subscript F indicates the Frobenius norm. When the γ_{p,q} value is close to 1, it indicates that the modal information redundancy between the two positions is very large, containing almost the same displacement modal information, and these two positions do not need to exist at the same time such that you need to delete a position. In actual operation, an appropriate redundancy threshold h is needed. If the redundancy coefficient is greater than the redundancy threshold h, the corresponding sensor location will be deleted.

Step 2.10: Add one sensor location from the remaining candidate sensor positions to join the existing position. Calculate the CIE(S) value, and the sensor location corresponding to the maximum value is selected.

Step 2.11: Delete the selected sensor location from the remaining candidate sensor positions. Calculate the redundancy coefficients of the remaining positions and the existing sensor locations, and delete the positions from the remaining candidate sensor positions corresponding to the coefficients exceeding the redundancy threshold h.

Step 2.12: Check the remaining positions, if there is no remaining position, continue to the next step; if there are remaining positions, return to Step 2.11.

Step 2.13: Get the final sensor placement and jump out of the loop.

The beneficial effects of the invention: The conditional information entropy based sensor placement method proposed by the invention can reduce the uncertainty of structural modal identification, and make the identified structural modal parameters more accurate. Through the proposed theory, the influences of the structural model error and measurement noise on the measured structural responses are effectively separated. The existing information entropy theory cannot calculate the uncertainty of the modal identification parameters in this case, because the Fisher information matrix is uncertain. Using the proposed conditional information entropy theory, the uncertainty of the identified modal parameters caused by model errors and measurement noise can be well quantified. Through the method proposed by the invention, the accuracy of modal identification is guaranteed. Moreover, the present invention can prevent adjacent sensors from containing repeated modal information by setting a redundancy threshold.

### DESCRIPTION OF DRAWINGS

FIG. 1 is the schematic diagram of the finite element model of a simply supported beam.

FIG. 2(a) is the sensor placement without considering the redundant modal information.

FIG. 2(b) is the sensor placement considering the redundant modal information with a redundancy threshold of 0.8.

### DETAILED DESCRIPTION

The present invention is further described below in combination with the technical solution.

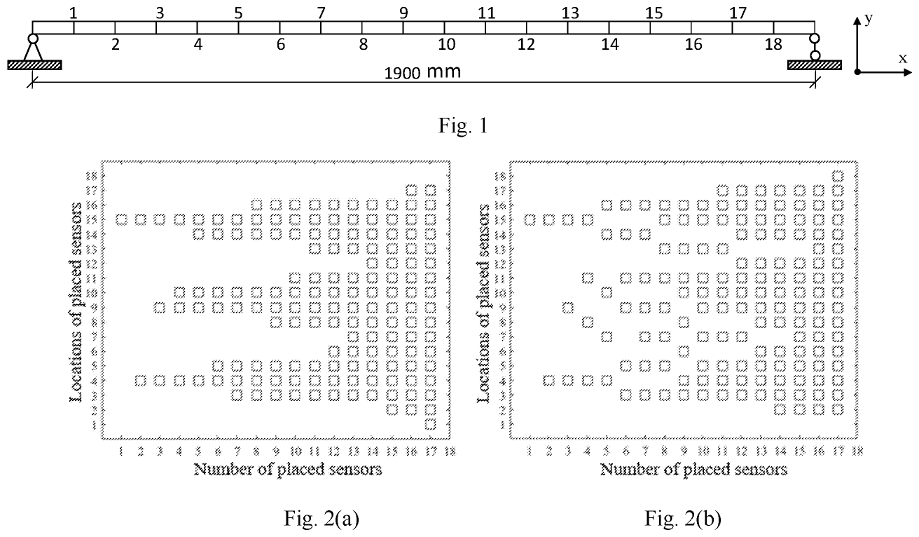

The sensor placement method uses a simply supported beam structure for simple verification. As shown in FIG. 1, the model consists of 19 two-dimensional Euler beam elements, each of which is 0.1 m long. Proportional damping is used so that the structure has the same mode shape matrix as the undamped case. The simply supported beam structure has 20 nodes and 57 degrees of freedom. The candidate sensor positions are the 18 vertical degrees of freedom. Here, the acceleration sensor, the velocity sensor and the displacement sensor can all be arranged using the sensor placement method proposed by the present invention.

(1) The finite element model is established, and the simply supported beam is divided into 20 nodes with 57 degrees of freedom. The 18 vertical vibration degrees of freedom are taken as the sensor candidate positions.

(2) The Monte Carlo method is used to derive B, the range of perturbation coefficients for the element stiffness matrices in each case. The perturbation coefficient β is set to a Gaussian random vector with a mean of 0. The covariance matrix is a diagonal matrix, and the diagonal elements are all 0.3.

(3) By Eq. (6), the mode shape matrix in each case are obtained: Φ+[E₁β, E₂β, . . . , E_{N}_{ₘ}β], β∈B.

(4) With steps 2.4 through 2.13 of the proposed conditional information entropy based sensor placement method, two final sensor placements are obtained in two cases: the redundancy threshold of 0.8 and without a redundancy threshold.

We claim:

1. A sensor placement method for reducing uncertainty of structural modal identification, wherein the steps are as follows: establish the relationship between structural model error and measurement noise; conditional information entropy based sensor placement method;
(1) establish the relationship between structural model error and measurement noise
step 1.1: In structural health monitoring systems, prediction error between the measured and actual structural response is due to two causes: model error and measurement noise, thereby establishing the following relationship:
y(t)=S(x(t,θ)+e(t,θ)) (1)
where: y(t)∈ [symbol] is a measured structural response of Ns degrees of freedom; N_{d} is a number of total degrees of freedom of the structure; S∈ [symbol] is a selection matrix for the sensor locations; θ∈ [symbol] is a modal parameter vector to be identified; e(t,θ)∈ [symbol] is a prediction error between the measured and actual structural response, which can be expressed as:
e(t,θ)=eₘₑₐ(t,θ)+e_{mod}(t,θ) (2)
where: eₘₑₐ(t,θ) is measurement noise; e_{mod}(t,θ) is the part of the prediction error caused by the structural model error;
step 1.2: define the form of the prediction error: the measurement noise is assumed to be a zero-mean Gaussian noise with a covariance matrix of Σₘₑₐ=diag(σ₁, . . . , σ_{N}_{_{d}}), σ₁=σ₀; the structural model error is represented by the stiffness variation of the structure which is shown as:
$$Δ K = {\sum }_{j = 1}^{{N}_{e}} {β}_{j} {K}_{j} \qquad ( 3 )$$
where: Nₑ represents a number of structural element stiffness matrices; Kⱼ is the jth element stiffness matrix; βⱼ is a perturbation coefficient of the jth element stiffness matrix;
change in the structural modal matrix is expressed as:
$$\begin{matrix}{ΔΦ}_{i} = [ {{\sum }_{r = 1}^{{N}_{d}}}_{r \neq i} \frac{- {Φ}_{r}^{T} {K}_{1} {Φ}_{i}}{{λ}_{r} - {λ}_{i}} {Φ}_{r} , … , {{\sum }_{r = 1}^{{N}_{d}}}_{r \neq i} \frac{- {Φ}_{r}^{T} {K}_{{N}_{e}} {Φ}_{i}}{{λ}_{r} - {λ}_{i}} {Φ}_{r} ] \\ {[ {β}_{1} , … , {β}_{{N}_{e}} ]}^{T} \\ = {E}_{i} β\end{matrix} \qquad ( 4 )$$
where: β is a perturbation coefficient vector of each element stiffness matrix, Eᵢ is a sensitivity coefficient matrix of the ith mode; ΔΦᵢ is a change of the ith mode shape vector; Φᵣ is a rth mode shape vector; λᵣ and λₗ are the eigenvalues of the rth and ith mode respectively; the superscript T indicates transposition;
the mode shape changes of each order of the structure are expressed as:
ΔΦ=[E₁β,E₂τ3, . . . ,E_{N}_{ₘ}β] (5)
where: ΔΦ represents mode shape changes of each order mode of the structure; the subscript Nₘ indicates a corresponding modal order;
step 1.3: establish a measurement data expression that comprehensively considers the structural model error and measurement noise; Eq. (1) is rewritten as:
y(t)=S((Φ+[E₁β,E₂β, . . . ,E_{N}_{ₘ}β])θ+eₘₑₐ(t,θ)) (6)
where: Φ is a mode shape matrix calculated by the finite element model used in the structure; as seen from Eq. (6), the prediction error between the measured response and the actual response is represented by the two parts caused by the model error and the measurement noise;
(2) conditional information entropy based sensor placement method
step 2.1: use the probability density function to represent the uncertainty of the recognition result of modal coordinate parameters:
$$\begin{matrix}p ( θ \vert {Σ}_{mea} , D , β ) = c \frac{1}{{( \sqrt{2 π } {σ}_{0} )}^{{NN}_{s}}} exp [ - \frac{{NN}_{s}}{2 {σ}_{0}^{2}} J ( θ \vert D , β ) ] π ( θ \vert β ) & ( 7 ) \\ J ( θ \vert {Σ}_{mea} , D , β ) = \frac{1}{{NN}_{s}} {\sum }_{k = 1}^{N} {[ {y}_{k} - Sx ( θ , k \vert β ) ]}^{T} [ {y}_{k} - Sx ( θ , k \vert β ) ] & ( 8 )\end{matrix}$$
where: p(θ|Σₘₑₐ, D,β) indicates conditional probability density function; π(θ|β) is a prior distribution of modal coordinate parameters θ; c is a constant, ensuring that the integral summation value of Eq. (7) is 1; N is total number of samples; k represents sampling time;
step 2.2: according to Eq. (8), the Fisher information matrix is obtained:
Q(S,θ₀|β)=(S(Φ+[E₁β,E₂β, . . . ,E_{N}_{ₘ}β]))^{T}(S(Φ+[E₁β,E₂β, . . . ,E_{N}_{ₘ}β])) (9)
where: Q(S, θ₀|β) is the Fisher information matrix;
step 2.3: derive the conditional information entropy for quantifying and calculating the uncertainty of the modal parameter identification;
h(S|Σₘₑₐ,D,B)|∫_{β∈B}−ln[det(Q(S,θ₀|β))]π(β)dβ (10)
where: h(S|Σₘₑₐ, D, B) is conditional information entropy; B is a range of the perturbation coefficients;
remove the negative sign to get a conditional information entropy index:
CIE(S)=∫_{β∈B} ln [det(Q(S,θ₀|β))]π(β)dβ (11)
step 2.4: establish the finite element model of structure, determine the candidate sensor placement positions; use Monte Carlo method to obtain the range of the perturbation coefficient B and the structural mode shape matrix in the corresponding situation; the initial number of selected sensors is 0;
step 2.5: whether to consider structural modal information redundancy: do not consider, continue to the next step; consider, jump to step 2.9;
step 2.6: add one sensor location from the remaining candidate sensor positions to join the existing positions; calculate the CIE(S) value, and the sensor location corresponding to the maximum value is selected;
step 2.7: from the remaining candidate sensor positions, delete the selected sensor location; check the remaining positions, if there is no remaining position, continue to the next step; if there are remaining positions, return to step 2.6;
step 2.8: get the final sensor placement and jump out of the loop;
step 2.9: if there are sensor locations too close, they contain similar structural modal information, resulting in redundancy of acquired structural modal information; introduce the concept of structural modal information redundancy as:
$${γ}_{p , q} = 1 - \frac{{‖ {Φ}_{p} - {Φ}_{q} ‖}_{F}}{{‖ {Φ}_{p} ‖}_{F} + {‖ {Φ}_{q} ‖}_{F}} \qquad ( 12 )$$
where: γ_{p,q} represents a redundancy coefficient between the pth node position and the qth node position in the finite element structure; the subscript F indicates the Frobenius norm; when the γ_{p,q} value is close to 1, it indicates that the modal information redundancy between the two positions is very large, containing almost the same displacement modal information, and these two positions do not need to exist at the same time such that you need to delete a position; in actual operation, an appropriate redundancy threshold h is needed; if the redundancy coefficient is greater than the redundancy threshold h, the corresponding sensor location will be deleted;
step 2.10: add one sensor location from the remaining candidate sensor positions to join the existing position; calculate the CIE(S) value, and the sensor location corresponding to the maximum value is selected;
step 2.11: delete the selected sensor location from the remaining candidate sensor positions; calculate the redundancy coefficients of the remaining positions and the existing sensor locations, and delete the positions from the remaining candidate sensor positions corresponding to the coefficients exceeding the redundancy threshold h;
step 2.12: check the remaining positions, if there is no remaining position, continue to the next step; if there are remaining positions, return to step 2.11;
step 2.13: get the final sensor placement and jump out of the loop.
